(19) United States
(12) Patent Application Publication
(10) Pub. No.: US 2019/0254873 A1
(43) Pub. Date: Aug. 22, 2019

(54) SYSTEMS AND METHODS FOR REDUCING INTRAOCULAR PRESSURE

(71) Applicant: Camras Vision, Inc., Research Triangle Park, NC (US)

(72) Inventors: Lucinda J. Camras, Durham, NC (US); Rolf Erik Ypma, Durham, NC (US); Walt H. Bassett, Durham, NC (US)

(21) Appl. No.: 16/281,601

(22) Filed: Feb. 21, 2019

Related U.S. Application Data
(60) Provisional application No. 62/633,158, filed on Feb. 21, 2018.

(51) Int. Cl. A61F 9/007; A61K 9/00

(52) U.S. Cl. CPC A61F 9/00781; A61F 9/0017; A61K 9/0051

(57) ABSTRACT

A device drains aqueous humor from an eye. The device includes an inlet conduit positioned at least partially within an anterior chamber. The device includes a housing coupled to the inlet conduit. The housing includes a cavity in fluid communication with the inlet conduit. The inlet conduit includes an inlet passageway allowing aqueous humor to flow from the anterior chamber to the cavity. The device includes an outlet conduit coupled to the housing at a proximal end and in fluid communication with the cavity. The outlet conduit includes an outlet passageway allowing the aqueous humor to flow from the cavity at the proximal end to an external ocular surface via an outlet opening at the distal end. The device includes an anti-clogging element coupled to the outlet conduit and configured to prevent or remove an obstruction of the outlet conduit.

### CROSS REFERENCE TO RELATED APPLICATIONS

This application claims priority to and the benefit of U.S. Provisional Application No. 62/633,158, filed Feb. 21, 2018, the contents of which are incorporated entirely herein by reference.

### FIELD OF THE INVENTION

Aspects of the present disclosure are generally directed to systems and methods employing implantable devices that drain aqueous humor from an anterior chamber of an eye to a location external to or distal from the anterior chamber and, more particularly, to systems and methods that prevent and/or remove the accumulation of obstructive material in such implantable devices.

### BACKGROUND

Glaucoma is a group of chronic optic nerve diseases and a leading cause of irreversible blindness. The major risk factor in glaucoma is elevated intraocular pressure due to improper drainage of aqueous humor from the eye. Reduction of intraocular pressure is the only proven treatment to stop the progression of vision loss due to glaucoma.

Standard glaucoma surgeries to reduce intraocular pressure, such as trabeculectomies and glaucoma drainage device implantation, tend to be lengthy and traumatic with unpredictable outcomes and complication rates of 20-60%. Implantable drainage devices operate to drain excess aqueous humor from the eye, and installation of such drainage devices typically requires a surgical opening to be made in the sclera to reach the interior of the eye, in particular the anterior chamber or the posterior chamber.

Dry eye disease is a common and complex condition in which the tear film layer is not properly maintained. Symptoms can range from intermittent and mild to a chronic, vision-threatening state. Tear production is similar to aqueous humor production and has a similar chemical composition.

Treatments for dry eye include topical drops, puncta occlusion, and gland stimulation treatments. Topical drops provide temporary relief and require frequent dosing. Punctal occlusion via plugs or cautery may be used to stop the uptake of tears by the puncta. However, punctal occlusion has poor retention rates and cautery is irreversible. Gland stimulation may also be used to increase tear break up time, but it is not effective in all patients and is a short-term solution. Therefore, there is an ongoing need to provide a therapy that provides continuous relief.

### SUMMARY

According to aspects of the present disclosure, systems and methods employ implantable devices that drain aqueous humor from an anterior chamber of an eye to a location external or distal to the anterior chamber. According to further aspects, the systems and methods prevent and/or remove the accumulation of obstructive material in such implantable devices. Such systems and methods may be employed to reduce intraocular pressure in order to treat glaucoma. Additionally or alternatively, such systems and methods may be employed to treat dry eye by directing the aqueous humor to the ocular surface where it can act as a lubricant.

According to one embodiment, a device for draining aqueous humor from an eye includes an inlet conduit configured to be positioned at least partially within an anterior chamber of an eye. The device includes a housing coupled to the inlet conduit. The housing defines a cavity that is in fluid communication with the inlet conduit. The inlet conduit includes an inlet passageway allowing aqueous humor to flow from the anterior chamber to the cavity. The device includes an outlet conduit extending from a proximal end to a distal end. The outlet conduit is coupled to the housing at the proximal end and in fluid communication with the cavity of the housing. The outlet conduit includes an outlet passageway allowing the aqueous humor to flow from the cavity at the proximal end to an external ocular surface via an outlet opening at the distal end. The device includes an anti-clogging element coupled to the outlet conduit and configured to prevent or remove an obstruction of the outlet conduit at the distal end caused by material from the external ocular surface.

According to another embodiment, a device for draining aqueous humor from an eye includes an inlet conduit configured to be positioned at least partially within an anterior chamber of an eye. The device includes a housing coupled to the inlet conduit. The housing includes a cavity that is in fluid communication with the inlet conduit. The inlet conduit includes an inlet passageway allowing aqueous humor to flow from the anterior chamber to the cavity. The device includes an outlet conduit extending from a proximal end to a distal end. The outlet conduit is coupled to the housing at the proximal end and in fluid communication with the cavity of the housing. The outlet conduit includes an outlet passageway allowing the aqueous humor to flow from the cavity at the proximal end to an external ocular surface via an outlet opening at the distal end. The outlet conduit is formed at least partially from a material having anti-fouling properties.

These and other aspects of the present disclosure will become more apparent from the following detailed description of embodiments of the present disclosure when viewed in conjunction with the accompanying drawings, which are briefly described below.

### BRIEF DESCRIPTION OF THE DRAWINGS

FIG. 1A illustrates aspects of an example drainage device, according to aspects of the present disclosure.

FIG. 1B illustrates aspects of another example drainage device, according to aspects of the present disclosure.

FIG. 2 illustrates an example embodiment of a drainage device, according to aspects of the present disclosure.

FIG. 3A illustrates an example embodiment and implementation of a drainage device, according to aspects of the present disclosure.

FIG. 3B illustrates another example embodiment and implementation of a drainage device, according to aspects of the present disclosure.

FIG. 4A illustrates a side cross-sectional view of an example outlet conduit of a drainage device where an example anti-clogging element is coupled to the outlet conduit, according to aspects of the present disclosure.

FIG. 4B illustrates a perspective cross-sectional view of the example outlet conduit and the example anti-clogging element illustrated in FIG. 4A.

FIG. 5A illustrates a side cross-sectional view of an example outlet conduit of a drainage device where another example anti-clogging element is coupled to the outlet conduit, according to aspects of the present disclosure.

FIG. 5B illustrates a perspective cross-sectional view of the example outlet conduit and the example anti-clogging element illustrated in FIG. 5A.

FIG. 6A illustrates a side cross-sectional view of an example outlet conduit of a drainage device where yet another example anti-clogging element is coupled to the outlet conduit, according to aspects of the present disclosure.

FIG. 6B illustrates a perspective cross-sectional view of the example outlet conduit and the example anti-clogging element illustrated in FIG. 6A.

FIG. 7A illustrates a perspective cross-sectional view of an example drainage device employing a trap to keep proteins from flowing to and clogging a filter, according to aspects of the present disclosure.

FIG. 7B illustrates a side cross-sectional of the example drainage device illustrated in FIG. 7A.

FIG. 8 illustrates a side cross-sectional view of an example drainage device employing another trap to keep proteins from flowing to and clogging a filter, according to aspects of the present disclosure.

FIG. 9 illustrates illustrates an example drainage device configured to allow clogging proteins to be flushed from a filter and/or inlet conduit, according to aspects of the present disclosure.

FIG. 10A illustrates an example drainage device that is separable into two sections to allow a clogged filter to be removed, according to aspects of the present disclosure.

FIG. 10B illustrates an alternative drainage device that is separable into two sections to allow a clogged filter to be removed, according to aspects of the present disclosure.

FIG. 11A illustrates a perspective view of an example drainage device including two flow paths defined in part by two outlet conduits, according to aspects of the present disclosure.

FIG. 11B illustrates a perspective cross-sectional view of the drainage device of FIG. 11A showing a filter in the two flow paths.

FIG. 11C illustrates a side cross-sectional view of the drainage device of FIG. 11A showing one of the flow paths.

FIG. 11D illustrates a top cross-sectional view of an example replacement of the filter for the drainage device of FIG. 11A.

FIG. 11E illustrates a perspective cross-sectional view of the replacement of the filter for the drainage device of FIG. 11A.

FIG. 12 illustrates an assembly view of an example drainage device that includes two outlet conduits associated with different respective flow paths that can be selected to allow flow through an unclogged region of a filter, according to aspects of the present disclosure.

FIG. 13 illustrates an assembly view of an example drainage device that includes two inlet conduits associated with different respective flow paths that can be selected to allow flow through an unclogged region of a filter, according to aspects of the present disclosure.

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein.

### DETAILED DESCRIPTION

Implantable drainage devices operate to drain excess aqueous humor from the eye, and installation of such drainage devices typically requires a surgical opening made in the sclera to reach the interior of the eye, in particular the anterior chamber or the posterior chamber. Some drainage devices, also known as subconjunctival shunts, can be inserted into the interior of the eye to conduct the aqueous humor to the subconjunctival space. A problem associated with subconjunctival shunts, however, involves potential scarring of the bleb in the subconjunctival space and fibrous capsule formation around the outlet, which in many cases requires surgical revision that leads to additional risk of complications.

Other drainage devices, also known as external shunts, drain aqueous humor from the interior of the eye externally to the conjunctiva. External shunts avoid bleb and fibrous capsule formation and the unpredictability of wound healing in the subconjunctival space. Often, however, parts of an external shunt especially those that lie on the corneal surface, may he perceived by the patient to be a foreign body. External shunts can also be displaced by local tissue motion or extruded by constrictive wound healing processes. Furthermore, conduits of external shunts can transmit microorganisms from the outside to the interior of the eye, potentially leading to retrograde infection.

Embodiments according to the present disclosure address the disadvantages of current drainage devices and provide an improved drainage device for directing aqueous humor away from the anterior chamber to a desired location external to or distal from the anterior chamber. The drainage device may be employed to treat glaucoma by reducing intraocular pressure. Additionally or alternatively, the drainage device may be employed to treat dry eye disease by directing the aqueous humor to lubricate the ocular surface.

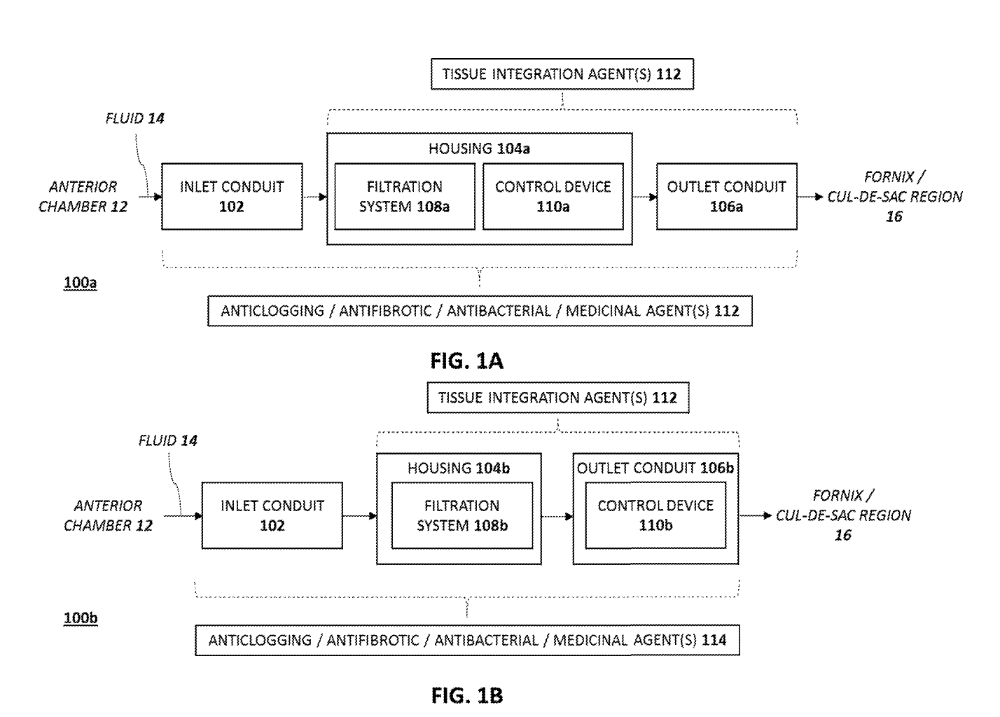

FIG. 1A illustrates aspects of an example drainage device 100a. The drainage device 100a includes an inlet conduit 102 that can be positioned at least partially within an anterior chamber 12 of an eye, so that aqueous humor 14 can flow from the anterior chamber 12 into the inlet conduit 102. The drainage device 100a also includes a housing 104a coupled to the inlet conduit 102. The housing 104a includes a cavity that is in fluid communication with the inlet conduit 102, and the inlet conduit 102 provides a passageway for the flow of the aqueous humor 14 from the anterior chamber 12 to the cavity. Additionally, the drainage device 100a includes an outlet conduit 106a coupled to the housing 104a. The outlet conduit 106a is in fluid communication with the cavity of the housing 204 and provides a passageway for the flow of the aqueous humor 14 from the cavity to an external ocular surface, e.g., a fornix/cul-de-sac region 16 underneath an eyelid.

To provide improved features for the drainage device 100a, tissue integration agents 112 may be applied to the housing 104a and/or the outlet conduit 106a, The tissue integration agents 112 can help position the housing 104a and/or the outlet conduit 106a more securely against surfaces/features of the eye when the drainage device 100a is implanted. Additionally or alternatively, other types of agents 114 may be applied to the inlet conduit 102, the housing 104a, and/or the outlet conduit 106a. Such agents 114, for instance, may include antimicrobial and/or medicinal agents.

As also shown in FIG. 1A, the drainage device 100a includes a filtration system 108a and a control device 110a that is disposed within the cavity of the housing 104a. When flowing through the cavity, the aqueous humor 14 flows through the filtration system 108a and the control device 110a. The control device 110a is configured to regulate the intraocular pressure within the drainage device 100a.

Creating a flow path from the anterior chamber to the external ocular surface with the drainage device 100a might raise concerns over the migration of microorganisms, such as bacteria, viruses, fungi, and spores, into the anterior chamber 12 and the corresponding risk of infection and inflammation. The filtration system 108a operates to prevent the upstream migration of microorganisms into the inlet conduit 102 and the anterior chamber 12, thereby reducing the likelihood of infection. Antimicrobial agents or materials may also be employed in the filtration system 108a. For instance, an antimicrobial coating may applied to aspects of the filtration system 108a or aspects of the filtration system 108a may be formed from materials including antimicrobial agents.

In some cases, the filtration system 108a includes a material with pores that are sufficiently small, e.g., less than approximately 0.4 μm, to prevent migration of microorganisms. For instance, the porous material may be a microporous/nanoporous membrane or polymer network, fiber network, or microcapsular material having a network of pores. In further cases, the pores may be arranged according to a gradient of pore sizes along the length of the filtration system 108a. For instance, the pores may be arranged so that the pore sizes continually decrease in the direction of flow from the inlet conduit 102 toward the outlet conduit 106a. The gradient of pore sizes can prevent debris accumulation and clogging within the filtration system 108a.

The control device 110a can provide resistance to achieve a particular rate for the flow of the aqueous humor 14 through the cavity of the housing 104a. The control device 110a may be removable and/or adjustable to achieve the particular flow rate and pressure gradient. In some cases, the filtration system 108a can also provide resistance to the flow of the aqueous humor 14.

FIG. 1B illustrates aspects of another example drainage device 100. Like the drainage device 100a above, the drainage device 100b includes the inlet conduit 102, which can be positioned at least partially within the anterior chamber 12 of an eye. The drainage device 100b also includes a housing 104b coupled to the inlet conduit 102. Like the housing 104a above, the housing 104b includes a cavity that is in fluid communication with the inlet conduit 102, and the inlet conduit 102 provides a passageway for the flow of the aqueous humor 14 into the cavity. Additionally, the drainage device 100b includes an outlet conduit 106b coupled to the housing 104b. The outlet conduit 106b is in fluid communication with the cavity of the housing and provides a passageway for the flow of the aqueous humor 14 from the cavity to the external ocular surface, e.g., the fornix/cul-de-sac region 16. The tissue integration agents 112 may be applied to the housing 104b and/or the outlet conduit 106b. Additionally or alternatively, other agents 114, such as antimicrobial and/or medicinal agents, may be applied to the inlet conduit 102, the housing 104b, and/or the outlet conduit 106b.

As also shown in FIG. 1B, the drainage device 100b includes a filtration system 108b and a control device 110b. Unlike the configuration of the drainage device 100a, however, the filtration system 108b is disposed within the cavity of the housing 104b, while the control device 110b is disposed within the outlet conduit 106b. When flowing through the cavity, the aqueous humor 14 flows through the filtration system 108a. When flowing through the outlet conduit 106b, the aqueous humor 14 flows through the control device 110b. Otherwise operating in a manner similar to the filtration system 108a, the filtration system 108b as well as antimicrobial agents operate to prevent upstream migration of microorganisms into the inlet conduit 102 and the anterior chamber 12. Meanwhile, the control device 110b is configured to regulate the intraocular pressure within the drainage device 100b, e.g., achieve a particular flow rate, by providing resistance to flow in the outlet conduit 106b.

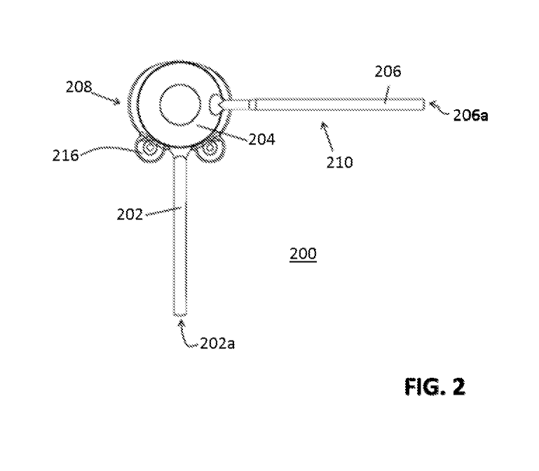

FIG. 2 illustrates an example embodiment of a drainage device 200. In particular, the drainage device 200 includes an inlet conduit 202 that can be positioned at least partially within the anterior chamber of the eye. The inlet conduit 202 includes an opening 202a at a proximal end through which aqueous humor from the anterior chamber can flow into the inlet conduit 202. The drainage device 200 includes a housing 204 coupled to a distal end of the inlet conduit 202. The housing 204 has a substantially circular disk-like shape and includes a cavity that is in fluid communication with the inlet conduit 202. As shown in FIG. 2, the inlet conduit 202 has an elongate tubular structure, which provides a passageway for the flow of the aqueous humor into the cavity.

The drainage device 200 includes a filtration system 208, which is disposed in the cavity of the housing 204. As described above, the filtration system 208 operates to prevent migration of microorganisms into the inlet conduit 202 and reduce the likelihood of reflux infection. The drainage device 200 also includes an outlet conduit 206. The housing 204 is coupled to a proximal end of the outlet conduit 206. As shown in FIG. 2, the outlet conduit 206 is an elongate tubular structure that extends from the housing 204 along an axis that is substantially perpendicular to the inlet conduit 202. The outlet conduit 206 is in fluid communication with the cavity of the housing 204. The outlet conduit 206 includes an opening 206a at a distal end and provides a passageway for the flow of the aqueous humor from the cavity through the opening 206a. The configuration of the drainage device 200 allows the opening 206a to be positioned at a desired location external to or distal from the anterior chamber, e.g., at the fornix/cul-de-sac region, where the flow of the aqueous humor can be directed. The desired positioning of the drainage device 200 relative to the eye can be achieved by employing eyelets 216 which accommodate the use of sutures for implantation.

The drainage device 200 also includes a control device 210, which is disposed in the outlet conduit 206. The control device 210 is configured to regulate the intraocular pressure within the drainage device 200. In particular, the control device 210 can provide resistance to achieve a particular rate for the flow of the aqueous humor through the outlet conduit 206. The control device 210 may be removable and/or adjustable to achieve the particular flow rate.

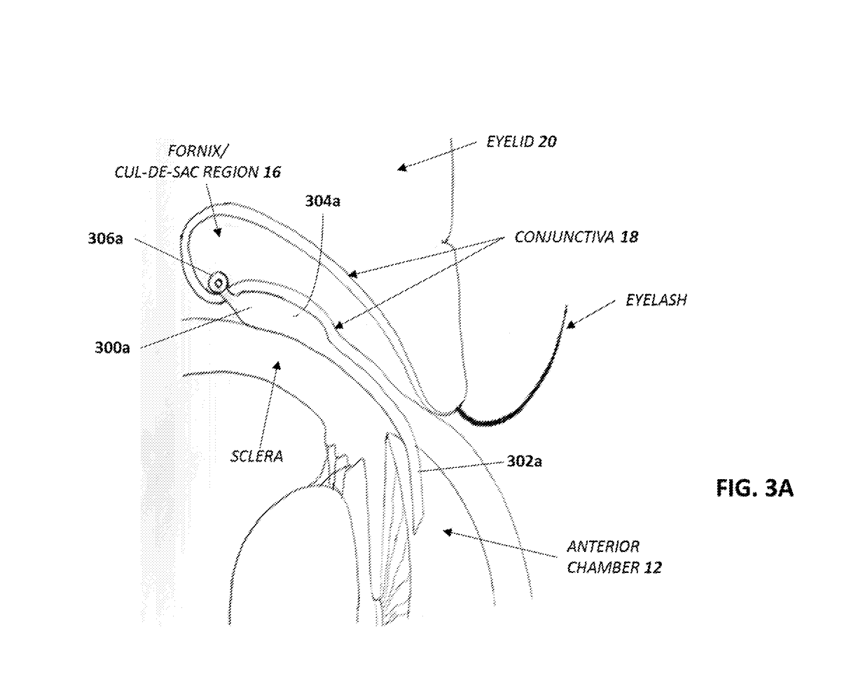

FIG. 3A illustrates an example embodiment and implementation of a drainage device 300a. Similar to the drainage devices above, the drainage device 300a includes an inlet conduit 302a, a housing 304a, and an outlet conduit 306a, where the inlet conduit 302a and the outlet conduit 306a are coupled to the housing 304a. The drainage device 300a is configured so that, when implemented as shown in FIG. 3A, the inlet conduit 302a can be positioned at least partially within the anterior chamber 12 and extend to the housing 304a. The drainage device 300a is further configured so that the housing 304a can be positioned under the conjunctiva 18 and the outlet conduit 306a can extend away from the housing 304a to an external position on the conjunctiva 18 in the fornix/cul-de-sac region 16, i.e., on the ocular surface and underneath the eyelid 20. Thus, the aqueous humor flows from the anterior chamber 12 into the inlet conduit 302a, passes though the housing 304a, and flows out of the outlet conduit 306a to a desired location external to or distal from the anterior chamber 12.

FIG. 3B illustrates another example embodiment and implementation of a drainage device 300b. Similar to the drainages devices above, the drainage device 300a includes an inlet conduit 302b, a housing 304b, and an outlet conduit 306b, where the inlet conduit 302b and the outlet conduit 306b are coupled to the housing 304b. The drainage device 300b is configured so that, when implemented as shown in FIG. 3B, the inlet conduit 302b can be positioned at least partially within the anterior chamber 12 and extend to the housing 304b. In contrast to the configuration of the drainage device 300a, however, the drainage device 300b is further configured so that the housing 304b as well as the outlet conduit 306b can be positioned externally on the conjunctiva 18 in the fornix/cul-de-sac region 16, i.e., on the ocular surface and underneath the eyelid 20. Thus, the aqueous humor flows from the anterior chamber 12 into inlet conduit 302b, passes though the housing 304b, and flows out of the outlet conduit 306b to a desired location external to or distal from the anterior chamber 12.

All drainage devices implanted in the eye have the potential to clog from proteins or other substances in the aqueous humor. Clogging reduces flow through a drainage device and may lead to elevation of intraocular pressure compared to baseline. Obstructive material may include endogenous material produced inside the eye or on the ocular surface, such as proteins and cells, and exogenous material, such as allergens, debris, or pathogens, that can foul one or more aspects of a drainage device. Specific embodiments disclosed herein prevent and/or remove the accumulation of obstructive material in such implantable devices. As described herein, preventing the accumulation of obstructive material can mean reducing, completely or by any other amount, the likelihood of any such accumulation.

In particular, the present inventors have identified an unanticipated problem involving the encapsulation of mucosubstances at the outlet conduit of the drainage devices described above. The encapsulation of mucosubstances results in the obstruction of the outlet conduit. Therefore, in some embodiments, anti-clogging elements are employed to prevent and/or remove the occurrence of such an obstruction of the outlet conduit.

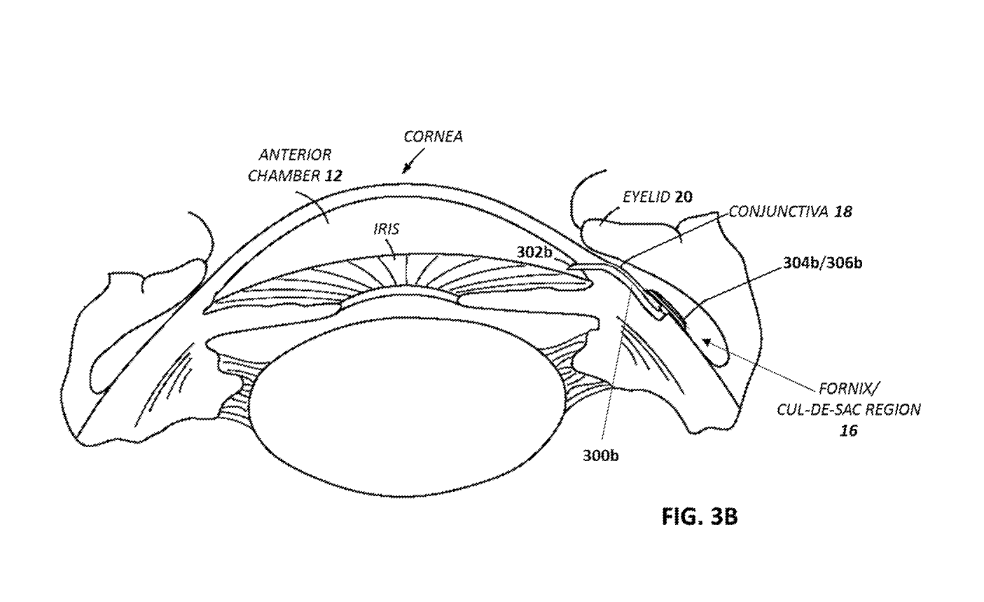

FIGS. 4A-B illustrate an example outlet conduit 406 of a drainage device where an example anti-clogging element 420 is coupled to the outlet conduit 406. FIG. 4A illustrates a side cross-sectional view, while FIG. 4B illustrates a perspective cross-sectional view. The outlet conduit 406 includes a wall 406a (e.g., a tubular wall) that defines an outlet passageway 406b that extends from a proximal end 406c to a distal end 406d. The outlet passageway 406b includes an outlet opening 406e at the distal end 406d. The outlet passageway 406b allows the aqueous humor to flow from the drainage device to an external ocular surface via the outlet opening 406e at the distal end 406d.

According to an embodiment, the outlet conduit 406 is coupled, at the proximal end 406c, to a housing of the drainage device. The housing device is also coupled to an inlet conduit which can be positioned at least partially within an anterior chamber of an eye. The housing includes a cavity that is in fluid communication with the inlet conduit. The inlet conduit includes an inlet passageway that allows aqueous humor to flow from the anterior chamber to the cavity of the housing. Meanwhile, the outlet passageway 406b of the outlet conduit 406 is in fluid communication with the cavity of the housing and allows the aqueous humor to flow from the cavity to the external ocular surface.

As shown further in FIGS. 4A-B, the anti-clogging element 420 includes a wicking material 422 which is disposed in the outlet passageway 406b of the outlet conduit 406 at the distal end 406d. The wicking material 422 can prevent the entry of clogging material from the external ocular surface through the outlet conduit 406 via the outlet opening 406a. The wicking material 416 can also encourage unidirectional flow of the aqueous humor through the outlet conduit 406 and onto the external ocular surface.

The wicking material 422 may be formed from biocompatible permeable or porous materials, including for instance polyhydroxyethylmethacrylate, polyurethane, polystyrene-co-isobutylene-co-styrene, polyethylene, polyacrylamide, polyvinyl alcohol, silicone, polycarbonate, polyethersulfone, polytetrafluoroethylene, or the like. In some cases, the wicking material 422 can be inserted into the outlet conduit 406 and can susbequently expand into a tight mechanical fit (e.g., seal) against the interior surface of the outlet conduit 406. For instance, the wicking material 422 can absorb water and expand into a tight mechanical fit. Alternatively, the wicking material 422 can be compressible so that it can be squeezed into the outlet conduit 406 and form a tight mechanical fit.

When an obstruction results from the clogging material captured by the wicking material 416, the wicking material 416 can be removed from the outlet conduit 406. As such, the obstruction caused by the clogging material can be easily removed while the drainage device remains implanted. The removed wicking material 416 can be replaced with a new wicking material to prevent additional material from flowing through the outlet conduit 406.

As FIGS. 4A-B also illustrate, the anti-clogging element 420 may optionally include a sheath 424. The sheath 424 includes a wall 424a (e.g., a tubular wall) that defines a sheath passageway 424b which can be filled with the wicking material 422. The sheath 424 can be inserted into, or removed from, the outlet passageway 406b via the outlet opening 406a. When disposed in the outlet conduit 406, the wall 424a of the sheath 424 is positioned against the interior surface of the outlet conduit 406, with the wicking material 422 disposed in the sheath passageway 424b. Combined, the wicking material 422 and the sheath 424 extend across the outlet opening 406e to prevent the entry of clogging material from the external ocular surface. The aqueous humor in the outlet passageway 406b flows through the wicking material 422. Advantageously, the sheath 424 facilitates the positioning of the wicking material 422 in the outlet passageway 406b at the distal end 406d. Additionally, the sheath 424 facilitates the removal and replacement of the wicking material 422 when an obstruction occurs. The sheath 424 can remain securely in the outlet conduit 406 when experiencing typical physiologic forces associated with use of the drainage device, but can be manually removed from the outlet conduit 406 with the intentional application of greater forces. The sheath 424 may be formed from materials, including for instance polyimide, polyether ether ketone, polytetrafluoroethylene, polyvinylidene fluoride, polyurethane, polymethyl methacrylate, polyetherimide, polypropylene, polycarbonate, polyethersulfone, silicone, nitinol, silver, gold, or the like.

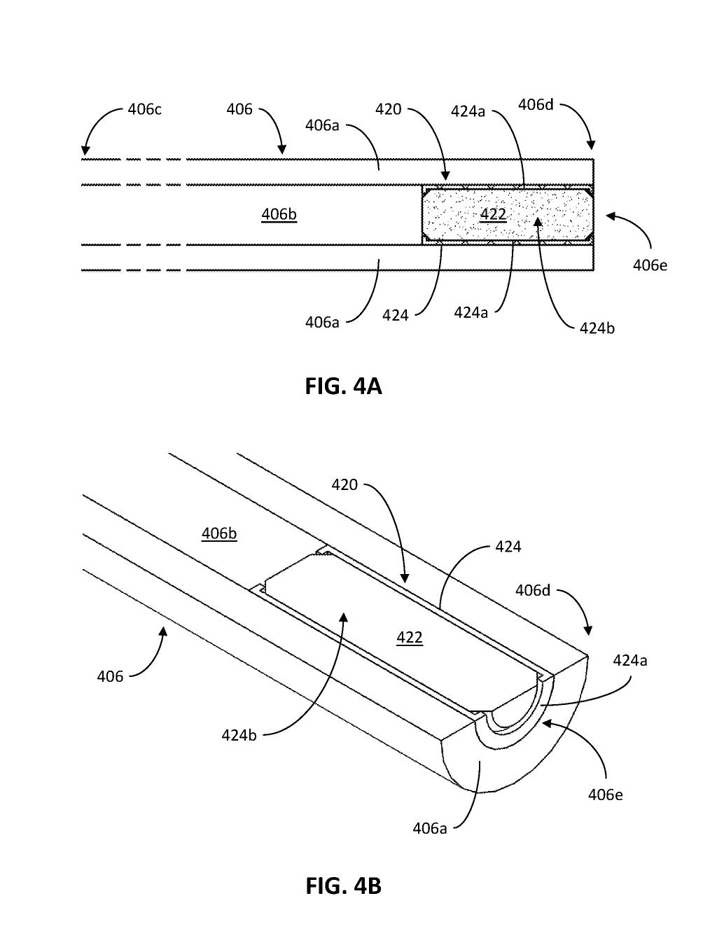

FIGS. 5A-B illustrate an example outlet conduit 506 of a drainage device where another example anti-clogging element 520 is coupled to the outlet conduit 506. FIG. 5A illustrates a side cross-sectional view, while FIG. 5B illustrates a perspective cross-sectional view. The outlet conduit 506 includes a wall 506a (e.g., a tubular wall) that defines an outlet passageway 506b that extends from a proximal end 506c to a distal end 506d. The outlet passageway 506b includes an outlet opening 506e at the distal end 506d. The outlet passageway 506b allows the aqueous humor to flow from the drainage device to an external ocular surface via the outlet opening 506e at the distal end 506d. Like the outlet conduit 406, the outlet conduit 506 may be coupled, at the proximal end 506c, to a housing of a drainage device as described above.

As shown further in FIGS. 5A-B, the anti-clogging element 520 includes a porous covering 522 (e.g., a sock) that is fitted over the outlet conduit 506 at the distal end 506d. The porous covering 522 includes a cavity 522a into which the outlet conduit 506 can be received. When fitted over the outlet conduit 506, the porous covering 522 allows passage of the aqueous humor from the outlet conduit 506. At the same time, the porous covering 522 prevents clogging material from the external ocular surface from entering the outlet passageway 506b via the outlet opening 506e.

When an obstruction results from material collected on the porous covering 522, the porous covering 522 can be removed from the outlet conduit 506. The obstruction caused by the material can be easily removed while the drainage device remains implanted. The removed porous covering 522 can be replaced with a new porous covering to prevent additional clogging material from flowing through the outlet conduit 506.

The porous covering 522 may be formed from an expandable material. As such, the porous covering 522 can be stretched to allow the porous covering 522 to slide onto or off the outlet conduit 506 more easily. Additionally, the expandable material also allows the porous covering 522 to engage the exterior surface of the outlet conduit 506 so that the porous covering 522 can remain securely over the outlet conduit 506 until intentional removal. The porous covering 522 may also be formed from softer materials that result in less irritation of ocular surfaces. Such materials, for instance, may include poly(2-hydroxyethyl methacrylate), poly vinyl alcohol, polyurethane, styrene isobutylene, or the like.

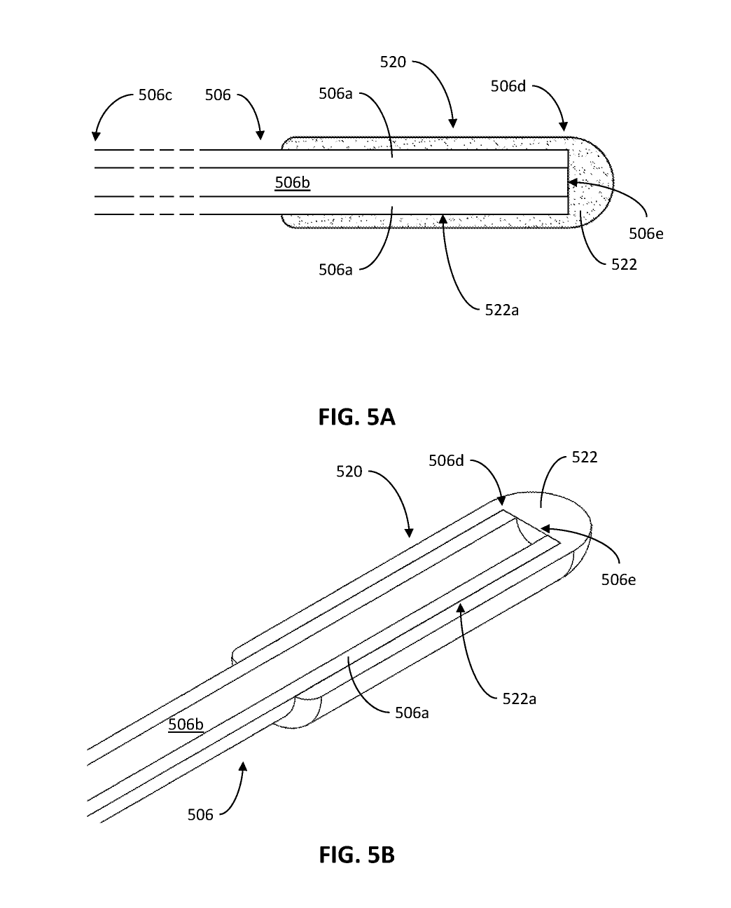

FIGS. 6A-B illustrate an example outlet conduit 606 of a drainage device where yet another example anti-clogging element 620 is coupled to the outlet conduit 606. FIG. 6A illustrates a side cross-sectional view, while FIG. 6B illustrates a perspective cross-sectional view. The outlet conduit 606 includes a wall 606a (e.g., a tubular wall) that defines an outlet passageway 606b that extends from a proximal end 606c to a distal end 606d. The outlet passageway 606b includes an outlet opening 606e at the distal end 606d. The outlet passageway 606b allows the aqueous humor to flow from the drainage device to an external ocular surface via the outlet opening 606e at the distal end 606d. Like the outlet conduits 406, 506, the outlet conduit 606 may be coupled, at the proximal end 606c, to a housing of a drainage device as described above.

As shown further in FIGS. 6A-B, the anti-clogging element 620 includes an inner conduit 622 that which is disposed in the outlet passageway 606b of the outlet conduit 606. The inner conduit 622 includes a wall 622a (e.g., a tubular wall) that defines an inner-conduit passageway 622b that extends from a proximal end 622c to a distal end 622d. The inner-conduit passageway 622b includes an inner-conduit opening 622e at the distal end 622d. When disposed inside the outlet conduit 606, the distal end 622d of the inner conduit 622 generally coincides with the distal end 606d of the outlet conduit 606. In addition, the inner conduit 622 is aligned with the outlet conduit 606 such that the aqueous humor flows in the outlet passageway 606b from the proximal end 606c of the outlet conduit 606 to the proximal end 622c of the inner conduit 622. The aqueous humor flows in the inner-conduit passageway 622b from the proximal end 622c to the distal end 622d. Because the inner conduit 622 is positioned at the distal end 606d of the outlet conduit 606, the aqueous humor exits the outlet conduit 606 to the external ocular surface as it flows through the inner-conduit opening 622e.

In addition, due to the position of the inner conduit 622 at the distal end 606d, any clogging material from the external ocular surface is collected in the inner-conduit passageway 622b. When an obstruction results from collected material, the inner conduit 622 can be removed from the outlet conduit 606. The obstruction caused by the material can be easily removed while the drainage device remains implanted. The removed inner conduit 622 can be replaced with a new inner conduit for further use of the drainage device.

The inner conduit 622 fits securely against the wall 606a of the outlet conduit 606 and remains in position when experiencing typical physiologic forces associated with use of the drainage device but can be manually removed from the outlet conduit 606 when required. The outlet passageway 606b may be shaped to accommodate the inner conduit 622. For instance, as illustrated in FIGS. 6A-B, the outlet passageway 606b may include a section of greater diameter to receive the inner conduit 622, which is shorter in length than the outlet conduit 606.

As described above, the anti-clogging elements 420, 520, 620 allow obstructions to be removed from a drainage device while the drainage device remains implanted. Aspects of the outlet conduits 406, 506, 606 and/or the anti-clogging elements 420, 520, 620 may be additionally formed from, or treated with, materials that resist fouling to reduce the likelihood of obstructions. The materials, for instance, may be resistant to fouling caused by bacteria and other microorganisms. Additionally, the materials may have low binding affinity to mucosubstances.

Encapsulation of mucosubstances can result in the obstruction of the outlet conduit 406, 506, 606. The production of mucosubstances is associated with an immune response to foreign material on a mucous membrane. Because mucosubstances are attracted to microbes, the use of antimicrobial materials can reduce the likelihood of fouling due to the production of mucosubstances.

In general, the use of antimicrobial agents, as well as anti-scarring, fibrinolytic, anti-coagulant, and anti-inflammatory agents, in the materials can reduce the likelihood of contamination and obstruction. In some embodiments, aspects of the drainage devices may employ materials that are impregnated, coated, or absorbed with anti-fouling agents. Such materials may include: RNA III inhibiting peptide (inhibits cell-cell communication, leading to prevention of their adhesion and virulence); ionized fluoroplastic coatings (resistant to bacterial adhesion); selenium, gold, and/or silver (prevents the normal buildup of bacteria, film, and deposits on lenses); polyethylene glycol (provides physical, chemical, and biological barriers to the nonspecific binding of proteins, bacteria, and fibroblast cells); polyelectrolyte (promotes protein and cell immobilization); and/or heparin,

In further embodiments, aspects of the drainage devices may employ anti-fouling materials including polyisobutylene-co-polyurethane or polystyrene-co-isobutylene-co-styrene. In other embodiments, aspects of the drainage devices may include anti-fouling materials that are block copolymers which develop a micro-morphology with soft segment and hard segment domains, where the micro-morphology may be preferably measured in domains of 100 nanometers or less. In yet other embodiments, aspects of the drainage devices may employ materials that are etched or textured to resist attachment by microorganisms and mucosubstances.

As described above, agents may be employed to promote tissue integration of a drainage device. In some embodiments, a porous material may be additionally or alternatively employed on appropriate aspects of a drainage device to enhance tissue integration in subconjunctival regions. Such tissue integration can also reduce production of mucosubstances.

As described above, the outlet conduit of a drainage device may include a proximal portion coupled to the housing and a distal portion including an outlet opening through which the aqueous humor exits to the external ocular surface. In alternative embodiments, the distal portion may be coupled to the proximal portion by a connector that is operable to allow removal and replacement of the distal portion. Such embodiments provide another approach for removing an obstruction in the distal portion of the outlet conduit. In some cases, the proximal portion is configured to extend from the housing to a conjunctival location where the distal portion can be accessed and removed.

Embodiments above are configured to address obstructions in the outlet conduit caused by clogging materials from the external ocular surface. According to other aspects of the present disclosure, drainage devices may include anti-clogging elements that can prevent obstructions in other parts of the drainage devices. For instance, embodiments that employ filtration systems risk obstruction over time, so anti-clogging elements may be employed to prevent or remove the occurrence of obstructions within the filtration systems.

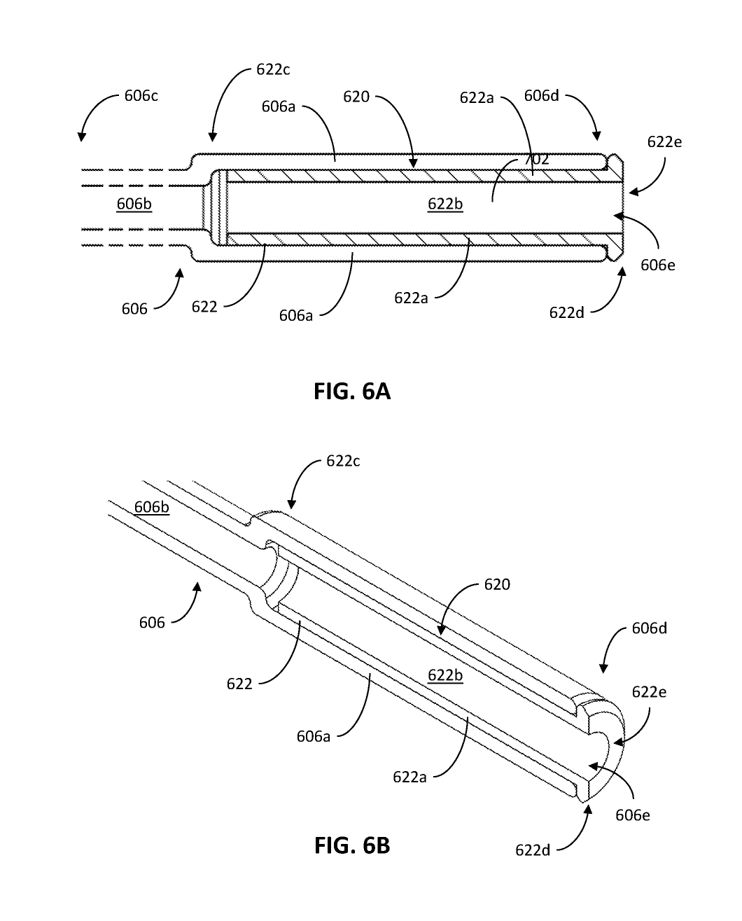

FIGS. 7A-B illustrate an example drainage device 700 including an inlet conduit 702, a housing 704, and an outlet conduit 706. FIG. 7A illustrates a perspective cross-sectional view, while FIG. 7B illustrates a side cross-sectional view. Similar to the embodiments described above, the inlet conduit 702 may be positioned at least partially within an anterior chamber of an eye. The housing 704 includes a cavity 704a that is in fluid communication with the inlet conduit 702, and the inlet conduit 702 provides a passageway for the flow of the aqueous humor from the anterior chamber to the cavity 704a. Additionally, the outlet conduit 706 is in fluid communication with the cavity 704a of the housing 704 and provides a passageway for the flow of the aqueous humor from the cavity 704a to an external ocular surface.

As also shown in FIGS. 7A-B, the drainage device 700 includes a filtration system, i.e., a filter 708, disposed in the cavity 704a of the housing 704. The filter 708 operates to prevent upstream migration of microorganisms into the inlet conduit 702 and thus the anterior chamber. For instance, the filter 708 may be formed from a material with pores that are sufficiently small, e.g., less than approximately 0.4 μm, to prevent the migration of microorganisms. The aqueous humor flows through filter 708. Proteins in the flow of aqueous humor can collect on the filter 708 and obstruct the small pores of the filter 708.

To slow the formation of any obstruction at the filter 708, the drainage device 700 also includes a trap 730 configured to keep proteins in the aqueous humor from flowing to the filter 708. As shown in FIGS. 7A-B, the trap 730 includes a surface 732 that is generally aligned with (e.g., parallel to) the flow of the aqueous humor from the inlet conduit 702 to the filter 708. The alignment of the surface 732 allows the aqueous humor to flow along the surface 732 without significant hindrance. As the flow of the aqueous humor passes over the surface 732, the surface 732 can attract the proteins in the flow of aqueous humor. For instance, the surface 732 may be formed from a hydrophobic material, such as polyethersulfone, polyether ether ketone, polyethylene, fluoropolymers (e.g., polytetrafluoroethylene, polyvinylidene fluoride), or the like. The proteins adhere to the surface 732 and thus kept from flowing to the filter 708.

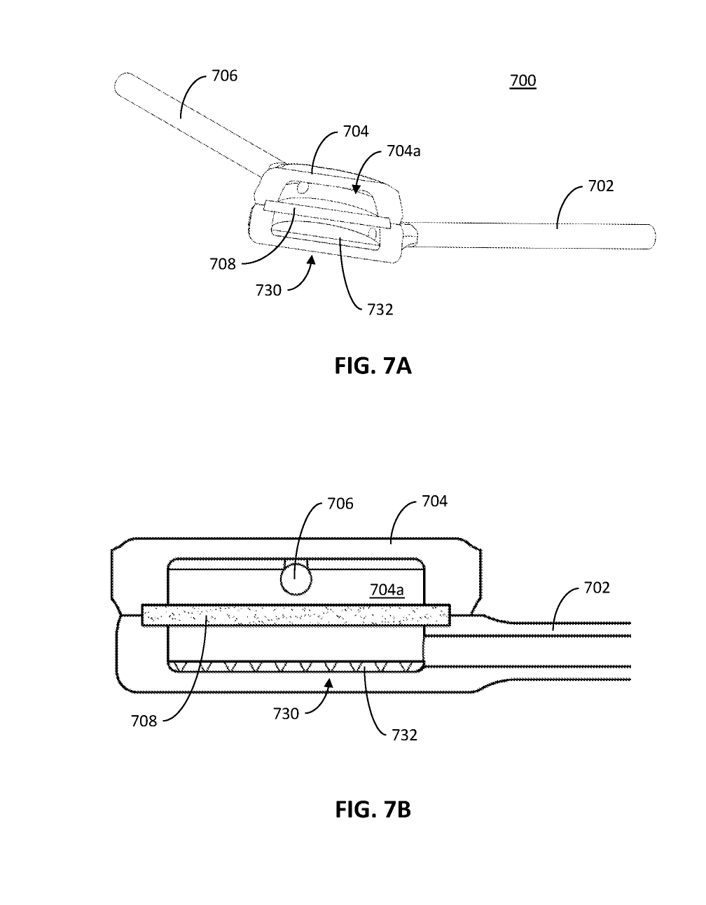

FIG. 8 illustrates an example drainage device 800 that includes an inlet conduit 802, a housing 804, an outlet conduit 806, and a filter 808, which are similar to those in the drainage device 700. The filter 808 is formed from a first porous material, e.g., with pores smaller than approximately 0.4 μm, to prevent the migration of microorganisms. The drainage device 800 is also configured to prevent proteins in the aqueous humor from flowing to the filter 808. In particular, the drainage device 800 employs a trap 830, which includes a screen 832 formed from a second porous material. For instance, the second porous material may include polyimide, polyether ether ketone, polytetrafluoroethylene, polyvinylidene fluoride, polyurethane, polycarbonate, polyethersulfone, polyhydroxyethylmethacrylate, polystyrene-co-isobutylene-co-styrene, polyethylene, polyacrylamide, polyvinyl alcohol, silicone, polycarbonate, polyethersulfone, or the like.

The screen 832 can collect the proteins in the flow before they reach the filter 808. The second porous material may include pores that are larger than pores of the first porous material of the filter 808, for instance, between approximately 0.4 μm to approximately 1000 μm.

Agents (e.g., anti-scarring, fibrinolytic, anti-coagulant, and anti-inflammatory agents) may be applied to the screen 832 to enhance anti-clogging characteristics. Some agents include those that combat fibroblast proliferation, which is involved in wound healing and contributes to scar formations (fibrosis). For instance, 5-fluorouracil is an agent that inhibits fibroblast proliferation. Other agents include mitomycin C. Yet other agents include collagenases which are enzymes that catalyze the hydrolysis of collagen and gelatin to prevent scarring. Another agent is heparin which has been used to coat intraocular lenses (IOLs) to reduce membrane formation. Heparin-sodium has been shown to reduce inflammation. Further agents include steroids such as triamcinolone or one of four essentially equivalent maximum-efficacy steroids: loteprednol etabonate 0.5%, 1% prednisolone acetate (Pred Forte), 1% prednisolone sodium phosphate, or 1% rimexolone for moderate to severe inflammation; and fluorometholones for mild to moderate inflammation.

In addition to reducing the likelihood of obstruction at the filter 808, the trap 830 may be further configured to change the fluid velocity profile of the flow of aqueous humor as it meets the filter 808. For instance, the screen 832 can modify the flow to have a more uniform velocity across the filter 808.

Once the filters 708, 808 become obstructed, the filters 708, 808 (or the drainage devices 700, 800 entirely) need replacement. Thus, by employing the traps 730, 830 to prevent obstruction at the filters 708, 808, the useful life of the filters 708, 808 can be prolonged.

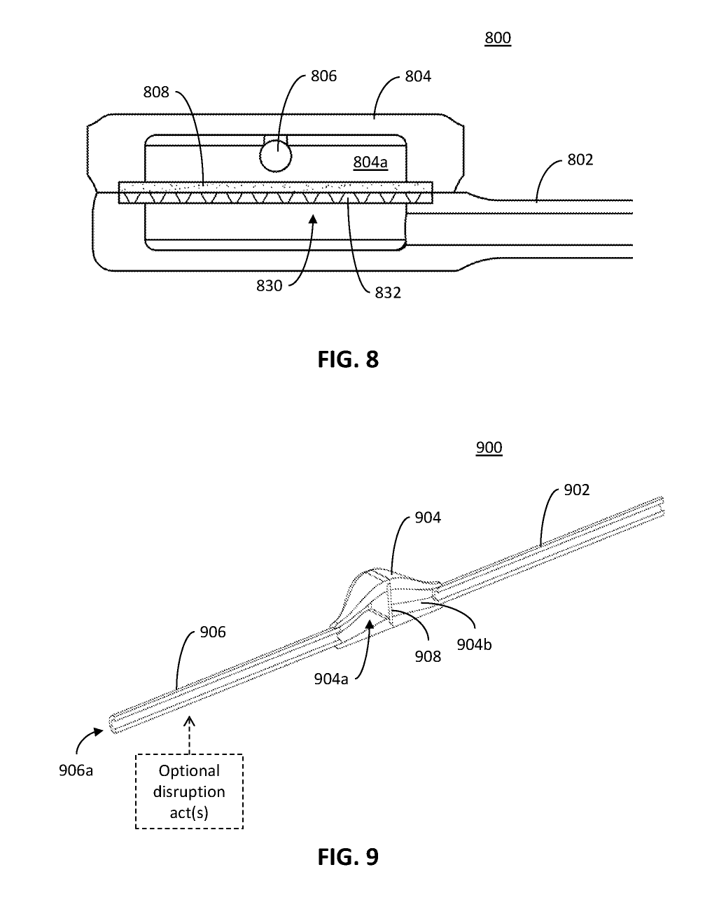

According to another approach, drainages devices may be configured to allow clogging proteins to be flushed from the filter and/or the inlet conduit. For instance, FIG. 9 illustrates an example drainage device 900 that includes an inlet conduit 902, a housing 904 with a cavity 904a, an outlet conduit 906, and a filter 908. Similar to the embodiments above, aqueous humor flows from the anterior chamber to an external ocular surface, via the inlet conduit 902, the cavity 904a, and the outlet conduit 906. The filter 908 is disposed in the cavity 904a and includes a porous material that operates to prevent migration of microorganisms into the inlet conduit 902 and thus the anterior chamber. As described above, proteins in the flow of aqueous humor can collect on the filter 908 and clog the filter 908.

To remove the clogging proteins from the filter 908, a flushing fluid can be delivered into the drainage device 900. The outlet conduit 906 includes an opening 906a where the aqueous humor flows out of the drainage device 900 to the external ocular surface. The opening 906a can also act as a two-way port to receive the flushing fluid. The flushing fluid flows through the outlet conduit 906 in a direction opposite to the flow of the aqueous humor. Thus, the flushing fluid flows into the cavity 904a and through the filter 908. As the flushing fluid flows through the filter 908, the flushing fluid can dislodge proteins that are clogging the filter 908. The flushing fluid then flows into the inlet conduit 902, carrying the dislodged protein from the filter 908.

As shown in FIG. 9, the housing 904 includes a funnel structure 904b in the cavity 904a between the filter 908 and the inlet conduit 902. The funnel structure 904b effectively receives the flushed material and guides the flushed material into the inlet conduit 902. In addition to removing the protein from the filter 908, the flushing fluid can also dislodge any proteins that are clogging the inlet conduit 902. The flushing fluid and the flushed material then flow into anterior chamber.

To allow the flushing fluid to dislodge the proteins more effectively, the filter 908 may be oriented to maximize the velocity of the flushing fluid at the filter 908. For instance, as shown in FIG. 9, the filter 908 may be perpendicular to the flow of the flushing fluid from the outlet conduit 906 to the inlet conduit 902. In other embodiments, the filter 908 may be diagonally oriented relative to the flow of the flushing fluid. In yet other embodiments, a component, such as a pin-wheel, upstream of the filter 908 may aid flushing by accelerating the velocity of the fluid out of the drainage device 900, where the component may accelerate fluid in response to an external magnetic field or the like.

Rather than employing the outlet opening 906a as a two-way port, alternative embodiments may employ the outlet opening 906a only to allow the aqueous humor to flow from the outlet conduit 906 and a separate flushing opening that receives the flushing fluid into the drainage device. In such embodiments, the first opening and the second opening each act as one-way ports.

Rather than allowing the flushing fluid to flow through the inlet conduit 902 to the anterior chamber, alternative embodiments may include a secondary flushing opening that allows the flushing fluid to flow out of the drainage device 900 into external region(s) or tissue(s) other than the anterior chamber. In some cases, a valve can control flow out of this secondary flushing opening. For instance, the secondary flushing opening may be positioned inside the cavity 904a of the housing 904, between the filter 908 and the inlet conduit 902. As such, the flushing fluid can flow from the drainage device 900 via the secondary flushing opening before reaching the inlet conduit 902. A valve may be employed to close the secondary flushing opening during normal operation of the drainage device, i.e., aqueous humor flows through the inlet conduit 902, the cavity 904a, and the outlet conduit 906 according to pressures within physiologic range. During flushing, however, the pressures associated with the flow of the flushing fluid exceed the physiologic range and cause the valve to open, thereby allowing the flushing fluid to flow through the secondary flushing opening rather than the inlet conduit 902. This mechanism allows for the clogging material to exit the device without over-pressurizing the device or eye by entering the anterior chamber.

In some embodiments, additional disruption act(s) may be optionally employed to disrupt (or break up) the clogging material on the filter 908 and/or in the inlet conduit 902 prior to applying the flushing fluid. For instance, a laser burst, sonication, ultrasound, heat, and/or a bioinert solution for dissolving the material may be directed at the clogging material.

In some cases, drainage devices may include microbeads that encapsulate proteases. The microbeads may be disposed at or near the filter 908 or the inlet conduit 902, e.g., in the cavity 904a between the filter 908 and the inlet conduit 902. A laser burst, sonication, ultrasound, heat, and/or other activating element may be directed to the microbeads to release the proteases and disrupt the clogging material.

In other cases, drainage devices may include an energy amplifying material, such as a piezoelectric material, disposed at or near the filter 908 or the inlet conduit 902. The energy amplifying material can amplify a sonic, ultrasound, mechanical, and/or electromagnetic signal applied to the filter 908 and/or the inlet conduit 902. The amplified signal enhances the disruption of the clogging material on the filter 908 and/or in the inlet conduit 902.

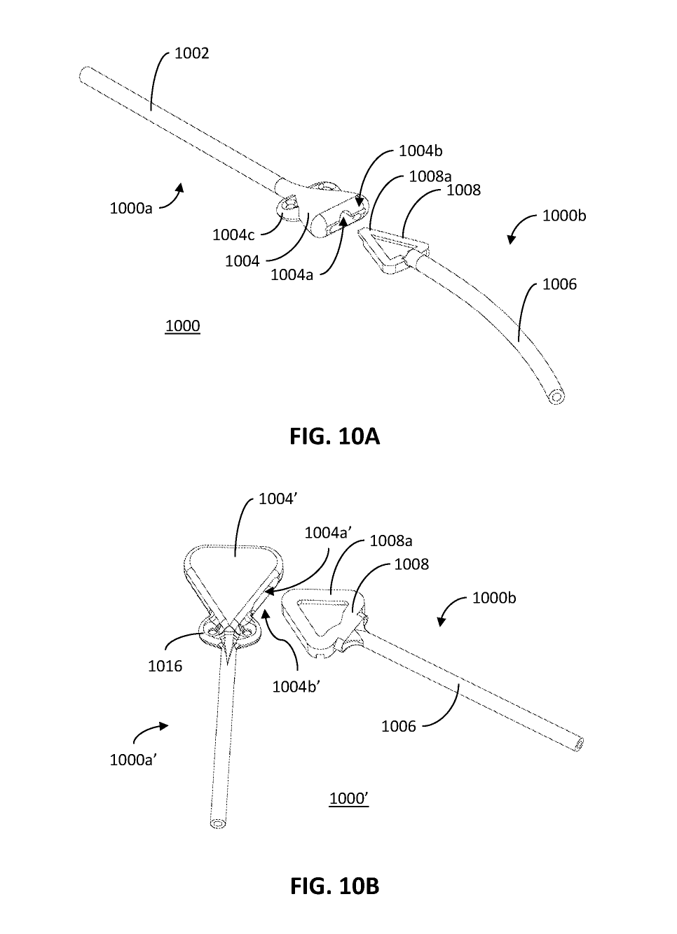

In embodiments above, elements of the drainage devices, e.g., the anti-clogging elements 420, 520, 620, can be removed and replaced to clear obstructions at the outlet conduit. In other embodiments, the filter can be removed and replaced to clear obstructions at the filter. For instance, FIG. 10A illustrates an example drainage device 1000 that includes an inlet conduit 1002, a housing 1004 with a cavity 1004a, an outlet conduit 1006, and a filter 1008. Similar to the embodiments above, aqueous humor flows from the anterior chamber to an external ocular surface, via the inlet conduit 1002, the cavity 1004a, and the outlet conduit 1006. The filter 1008 is disposed in the cavity 1004a and operates to prevent migration of microorganisms to the anterior chamber. As described above, proteins in the flow of aqueous humor can collect on the filter 1008 and cause obstructions at the filter 1008. The drainage device 1000 allows the filter 1008 to be removed to clear such obstructions from the drainage device 1000.

As FIG. 10A illustrates, the drainage device 1000 is defined by two separable sections 1000a, b. The first section 1000a includes the inlet conduit 1002 coupled to the housing 1004, and the second section 1000b includes the filter 1008 coupled to the outlet conduit 1006. The filter 1008 includes a casing 1008a that acts as a connecting structure that can be inserted into the cavity 1004a via a housing opening 1004b to connect the first section 1000a and the second section 1000b. The casing 1008a is sufficiently rigid to allow such insertion. When the first section 1000a and the second section 1000b are connected in this manner, the filter 1008 is positioned in the cavity 1004a. Additionally, and the inlet conduit 1002, the cavity 1004a, the filter 1008, and the outlet conduit 1006 are aligned to allow aqueous humor to flow through these elements.

In the example shown in FIG. 10A, the casing 1008a has a triangular shape. Correspondingly, the cavity 1004a has a complementary shape for receiving the casing 1008a. The triangular shape allows the casing 1008a to be easily guided into the cavity 1004a. The mechanical fit between the cavity 1004a and the casing 1008a creates a tight seal.

To remove and replace the filter 1008, the casing 1008a can be withdrawn from the cavity 1004a. For instance, aspects of the housing 1004 may be formed from a flexible material that allows it to become distorted (e.g., twisted, stretched, etc.) to accommodate removal/insertion of the casing 1008a. The outlet conduit 1006, which is accessible from outside the eye, can be manipulated to distort the casing 1008a and modify the fit between the casing 1008a and the cavity 1004a. The resulting distortion allows the casing 1008a to pulled from the cavity 1004a. Alternatively, the drainage device 1000 may include a release plug that can be operated to modify the fit and allow the casing 1008a to be pulled from the cavity 1004a. Or alternatively, the casing 1008a can be broken with laser, ultrasound, or the like to allow the casing 1008a to be pulled from the cavity 1004a.

Once the casing 1008a is withdrawn from the cavity 1004a, the second section 1000b can be separated from the first section 1000a, while the first section 1000a remains implanted in the eye. Indeed, as shown in FIG. 10A, the housing 1004 includes eyelets 1016 for sutures that allow the first section 1000a to be more permanently implanted in the eye than the second section 1000b. Meanwhile, the filter 1008 can be removed and replaced in the second section 1000b with a new filter, and the second section 1000b can be reconnected to the implanted first section 1000a to allow further use of the drainage device 1000. During filter replacement, a resistance mechanism, such as a valve or a collapsible conduit, may be employed (e.g., upstream of the filter) to prevent over-drainage which may lead to hypotony and to prevent contamination by microorganisms.

Alternatively, the filter 1008 can be treated to remove the clogging material, and the second section 1000b can be reconnected to the implanted first section 1000a to allow further use of the drainage device 1000. Alternatively, the second section 1000b with the clogged filter 1008 can be replaced with a new second section, and the new second section can be connected to the implanted first section 1000a to allow further use of the drainage device 1000.

In FIG. 10A, the first section 1000a and the second section 1000b are connected so that the inlet conduit 1002 and the outlet conduit 1006 are generally aligned along a common longitudinal axis. FIG. 10B, however, illustrates an alternative drainage device 1000′ with an alternative first section 1000a′. The alternative first section 1000a′ includes an alternative housing 1004′ coupled to the inlet conduit 1002. The alternative housing 1004′ includes an alternative cavity 1004a′ with an alternative opening 1004b′ that receives the casing 1008a of the second section 1000b according to a different orientation. As such, the alternative first section 1000a′ is connected to the second section 1000b so that the inlet conduit 1002 is generally perpendicular to the outlet conduit 1006. In other words, the inlet conduit 1002 and the outlet conduit 1006 are not aligned along the common longitudinal axis as shown in FIG. 10A. In general, the first section and the second section of a drainage device can be connected to orient the outlet conduit relative to the inlet conduit at any angle suitable for directing aqueous humor to a desired external ocular surface.

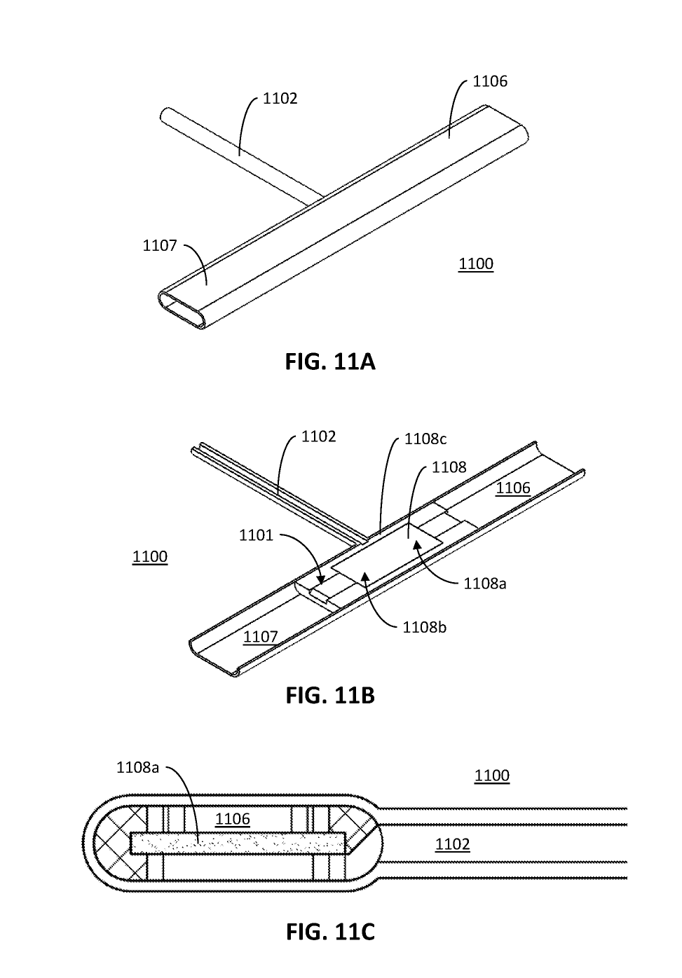

Embodiments may also include more than one inlet conduit or more than one outlet conduit. Such embodiments provide approaches for addressing clogging. For instance, FIGS. 11A-C illustrate an example drainage device 1100 that includes an inlet conduit 1102, a filter 1108, and two outlet conduits 1106, 1107. FIG. 11A illustrates a perspective view: FIG. 11B illustrates a perspective cross-sectional view; and FIG. 11C illustrates a side cross-sectional view.

As shown in FIG. 11B, the drainage device 1100 includes a first flow path and a second flow path. The first flow path includes the inlet conduit 1102, a first section 1108a of the filter 1108, and the first outlet conduit 1106. The second flow path includes the inlet conduit 1102, a second section 1108b of the filter 1108, and the second outlet conduit 1107. Initially, the second flow path is closed with a removable plug 1101, which prevents flow to the second outlet conduit 1107. As a result, the drainage device 1100 drains aqueous humor via the first flow path. FIG. 11C illustrates the first flow path of aqueous humor from the inlet conduit 1102, through the filter 1108, and into the first outlet conduit 1106.

As described above, proteins in the flow of aqueous humor can collect on the filter 1108 and cause obstructions at the filter 1108. For the first flow path, such obstructions occur in the first section 1108a of the filter 1108. When the drainage device 1100 cannot function effectively due to obstructions in the first section 1108a, the plug 1101 can be removed to open the second flow path. With the opening of the second flow path, aqueous humor can flow alternatively through the second section 1108b of the filter 1108 and the second outlet conduit 1107. Thus, the second flow path provides a backup flow path in case the first flow path becomes clogged.

The filter 1108 may include a casing 1108c, which allows the filter 1108 to be removed and replaced when clogged. For instance, as shown in FIGS. 11D-E, if the filter 1108 is clogged, a replacement filter 1108′ can be manually inserted into the second outlet conduit 1107 and pushed into the position of the clogged filter 1108. As the replacement filter 1108′ slides into the position, the replacement filter 1108′ pushes the clogged filter 1108 through the first outlet conduit 1106 where it can be taken out of the drainage device 1100.

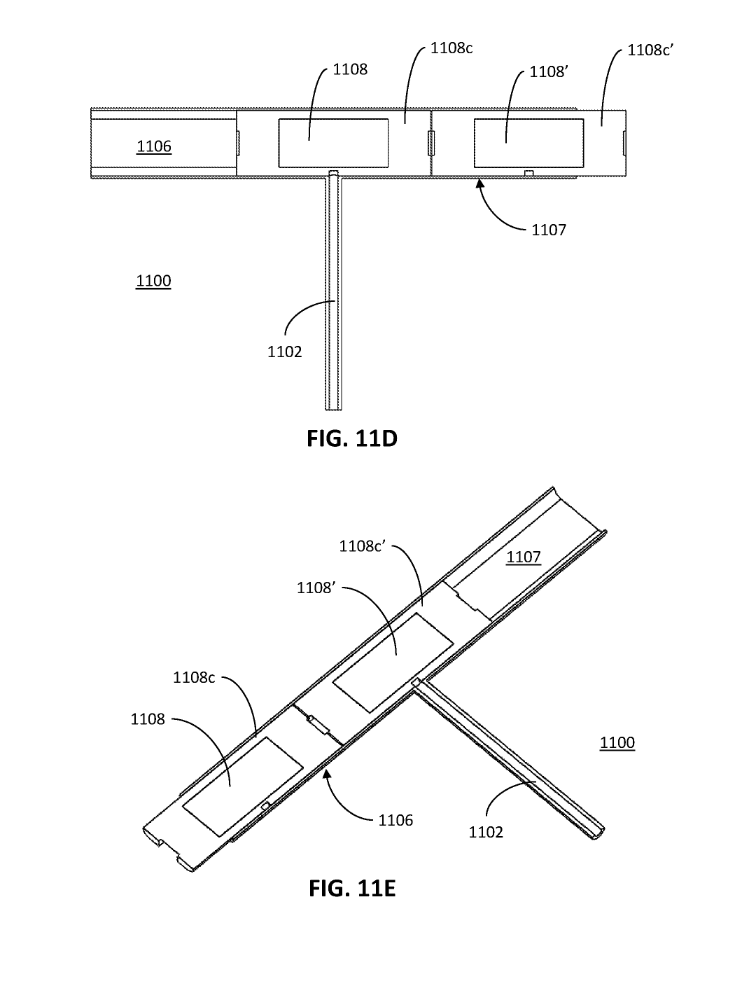

FIG. 12 illustrates an assembly view for an example drainage device 1200 that also includes two outlet conduits 1206, 1207. Additionally, the drainage device 1200 includes an inlet conduit 1202, a housing 1204, and a filter 1208. The filter 1208 is disposed in the housing 1204. The cavity in the housing 1204 is divided into two separate chambers 1204a, b between the filter 1208 and the outlet conduits 1206, 1207. The first outlet conduit 1206 extends from the first chamber 1204a, and the second outlet conduit 1207 extends from the second chamber 1204b. The first chamber 1204a is positioned over a first section of the filter 1208. The second chamber 1204b is positioned over a second section of the filter 1208. Thus, the drainage device 1200 includes a first flow path and a second flow path. The first flow path includes the inlet conduit 1202, the first section of the filter 1208, the first chamber 1204a and the first outlet conduit 1206. The second flow path includes the inlet conduit 1202, the second section of the filter 1208, the second chamber 1204b, and the second outlet conduit 1207.

Initially, a first plug 1201a may block the first outlet conduit 1206 and a second plug 1201b may block the second outlet conduit 1207. The first plug 1201a may be removed to allow the drainage device 1200 to drain aqueous humor via the first flow path, while the second plug 1201b remains in place to keep the second flow path closed. As described above, proteins in the flow of aqueous humor can collect on the filter 1208 and cause obstructions at the filter 1208. For the first flow path, such obstructions occur in the first section of the filter 1208. When the drainage device 1200 cannot function effectively due to obstructions in the first section, the plug can be selectively dissolved by a laser burst, ultrasound, heat, a bioinert solution, or the like to open the second flow path. This allows the drainage device 1200 to drain aqueous humor alternately via the second flow path. The second flow path provides a backup flow path in case the first flow path becomes clogged.

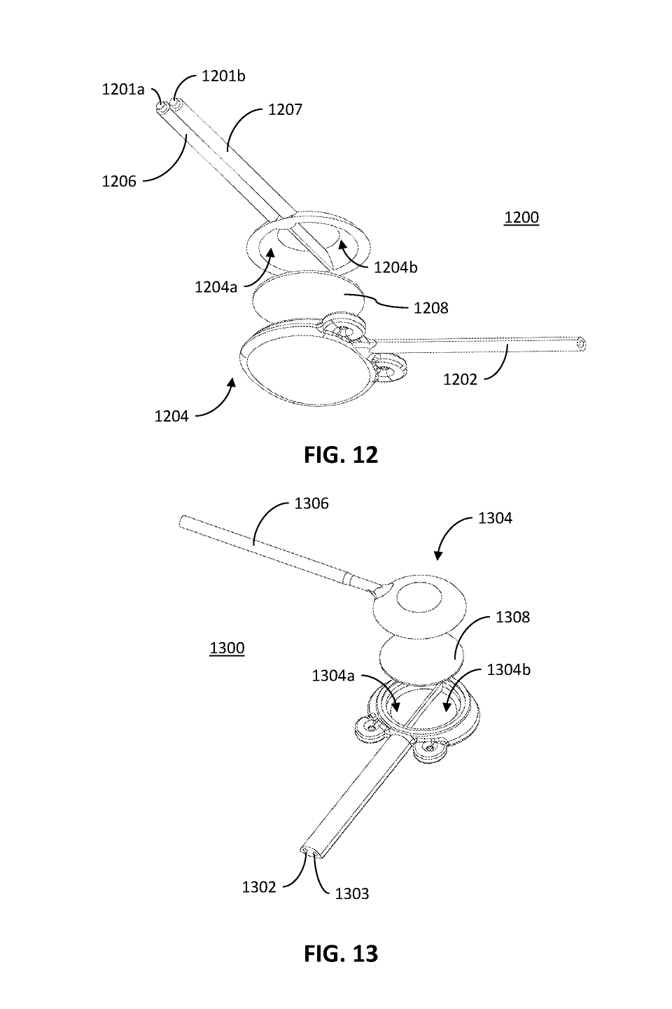

Alternatively, FIG. 13 illustrates an assembly view for an example drainage device 1300 that includes two inlet conduits 1302, 1303 as well as a housing 1304, a filter 1308, and an outlet conduit 1306. The filter 1308 is disposed in the housing 1304. The cavity in the housing 1304 is divided into two separate chambers 1304a, b between the filter 1308 and the inlet conduits 1302, 1303. The first inlet conduit 1302 leads to the first chamber 1304a, and the second inlet conduit 1303 leads to the second chamber 1304b. A first section of the filter 1308 is positioned over the first chamber 1304a. A second section of the filter 1308 is positioned over the second chamber 1304b. Thus, the drainage device 1300 includes a first flow path and a second flow path. The first flow path includes the first inlet conduit 1302, the first chamber 1304a, the first section of the filter 1308, and the outlet conduit 1306. The second flow path includes the second inlet conduit 1303, the second chamber 1304b, the second section of the filter 1308, and the outlet conduit 1306.

Initially, a plug may block the opening to the second inlet conduit 1303 to keep the second flow path closed. As such, the drainage device 1300 drains aqueous humor via the first flow path. As described above, proteins in the flow of aqueous humor can collect on the filter 1308 and cause obstructions at the filter 1308. For the first flow path, such obstructions occur in the first section of the filter 1308. When the drainage device 1300 cannot function effectively due to obstructions in the first section, the plug can be selectively dissolved by a laser burst, ultrasound, heat, a bioinert solution, or the like to open the second flow path. This allows the drainage device 1300 to drain aqueous humor alternately via the second flow path. Thus, the second flow path provides a backup flow path in case the first flow path becomes clogged.

In general, as shown in FIGS. 12-13, a drainage device may include a housing with multiple chambers that are aligned with different respective regions of a filter and connected to different respective inlet and/or outlet conduits. The inlet and/or the outlet conduits can be selectively opened or closed to create flow paths through different regions of the filter. When a particular region of the filter becomes clogged, a different flow path can be selected to allow aqueous humor to flow through a different (unclogged) region of the filter. For instance, a conduit can be plugged by a material that can be selectively dissolved by a laser burst, ultrasound, heat, a bioinert solution, or the like.

Alternatively, rather than blocking or opening conduits to select a flow path through an unclogged region of a filter, material may be applied to regions of the filter and the material may be selectively removed to allow flow through one or more particular unclogged regions. The material may be selectively dissolved by a laser burst, ultrasound, heat, a bioinert solution, or the like. In some embodiments, the material may be applied as a sheath over the filter and the sheath can be moved via a magnet to open flow through a region of the filter.

Alternatively, a drainage device may include a housing with multiple chambers that are separated by barriers and aligned with different respective regions of a filter. The barriers, however, include valves that can open channels through the barriers and allow fluid to flow between the chambers. For instance, the drainage device may initially employ a first flow path where aqueous humor flows from an inlet conduit into a first chamber and through a corresponding first region of the filter. If the first region of the filter becomes clogged, the pressure in the first chamber increases. In response to the increased pressure, a valve may open a channel between the first chamber and a second chamber. As such, the aqueous humor can flow from the first chamber to the second chamber. The second chamber is aligned with a second region of the filter. If the second region is unclogged, the aqueous humor can flow from the second chamber and through the second region. If the second region is or becomes clogged and the pressure in the second chamber increases, another valve may respond by opening a channel between the second chamber and a third chamber to allow flow through a third region of the filter, and so on.

Accordingly, aspects of the present disclosure provide approaches for preventing and/or removing the accumulation of obstructive material in different parts or aspects of implantable drainage devices.

While the present disclosure has been described with reference to one or more particular embodiments, those skilled in the art will recognize that many changes may be made thereto without departing from the spirit and scope of the present disclosure. Each of these embodiments and obvious variations thereof is contemplated as falling within the spirit and scope of the invention. It is also contemplated that additional embodiments according to aspects of the present disclosure may combine any number of features from any of the embodiments described herein.

What is claimed is:

1. A device for draining aqueous humor from an eye, comprising:
an inlet conduit configured to be positioned at least partially within an anterior chamber of an eye;
a housing coupled to the inlet conduit, the housing including a cavity that is in fluid communication with the inlet conduit, the inlet conduit including an inlet passageway allowing aqueous humor to flow from the anterior chamber to the cavity;
an outlet conduit extending from a proximal end to a distal end, the outlet conduit being coupled to the housing at the proximal end and in fluid communication with the cavity of the housing, the outlet conduit including an outlet passageway allowing the aqueous humor to flow from the cavity at the proximal end to an external ocular surface via an outlet opening at the distal end; and
an anti-clogging element coupled to the outlet conduit and configured to prevent or remove an obstruction of the outlet conduit at the distal end caused by material from the external ocular surface.

2. The device of claim 1, wherein the anti-clogging element includes a wicking material replaceably disposed in the outlet conduit at the distal end, the wicking material configured to draw the aqueous humor from the proximal end to the distal end and to prevent the material on the external ocular surface from entering the outlet conduit.

3. The device of claim 2, wherein the anti-clogging element further includes a sheath, the wicking material is disposed within the sheath, and the sheath is configured to be removably inserted into the outlet conduit via the outlet opening to allow positioning of the wicking material.

4. The device of claim 2, wherein the anti-clogging element is formed from an expandable material that allows the anti-clogging element to expand within the outlet conduit.

5. The device of claim 1, wherein the anti-clogging element includes a porous covering replaceably disposed over the outlet conduit at the distal end, the porous covering configured to allow passage of the aqueous humor from the outlet conduit through the porous covering and to prevent the material on the external ocular surface from entering the outlet conduit.

6. The device of claim 5, wherein the porous covering is formed from an expandable material that allows the porous covering to be expand over the outlet conduit.

7. The device of claim 1, wherein the anti-clogging element includes an inner conduit replaceably disposed in the outlet passageway of the outlet conduit, the inner conduit being configured to be removably inserted into the outlet conduit via the outlet opening.

8. The device of claim 1, further comprising:
a filtration system disposed in the housing and configured to filter microorganisms from migrating into the inlet conduit; and
a trap configured to trap proteins in the aqueous humor from flowing from the inlet conduit to the filtration system.

9. The device of claim 8, wherein the filtration system includes a first porous material configured to filter the microorganisms from migrating into the inlet conduit, and the trap includes a second porous material configured to filter the proteins in the aqueous humor from flowing to the filtration system, the second porous material including pores that are larger than pores of the first porous material.

10. The device of claim 8, further comprising an anti-fouling agent applied to the trap.

11. The device of claim 8, wherein the trap includes a surface aligned parallel to the flow of the aqueous humor to the filtration system and configured to cause the proteins in the aqueous humor to adhere to the surface.

12. The device of claim 8, wherein the trap is configured to modify a fluid velocity profile for the flow of the aqueous humor.

13. The device of claim 1, wherein the at least one of the outlet conduit or the anti-clogging element includes a material having anti-fouling properties.

14. The device of claim 13, wherein the anti-fouling material includes at least one of selenium, gold, silver, or heparin.

15. The device of claim 13, wherein the anti-fouling material includes polyisobutylene-co-polyurethane or polystyrene-co-isobutylene-co-styrene.

16. The device of claim 13, where in the anti-fouling material is a block copolymer that develops a micro-morphology including soft segment and hard segment domains.

17. The device of claim 16, wherein the micro-morphology is measured in domains of 100 nanometers or less.

18. The device of claim 13, wherein the anti-fouling material is etched or textured to resist attachment by microorganisms or mucosubstances.

19. The device of claim 1, further comprising a porous material applied to the housing or the outlet conduit, the porous material configured to enhance integration between the device and tissue.

20. A device for draining aqueous humor from an eye, comprising:
an inlet conduit configured to be positioned at least partially within an anterior chamber of an eye;
a housing coupled to the inlet conduit, the housing including a cavity that is in fluid communication with the inlet conduit, the inlet conduit including an inlet passageway allowing aqueous humor to flow from the anterior chamber to the cavity; and
an outlet conduit extending from a proximal end to a distal end, the outlet conduit being coupled to the housing at the proximal end and in fluid communication with the cavity of the housing, the outlet conduit including an outlet passageway allowing the aqueous humor to flow from the cavity at the proximal end to an external ocular surface via an outlet opening at the distal end, wherein the outlet conduit is formed at least partially from a material having anti-fouling properties.

21. The device of claim 20, wherein the anti-fouling material includes at least one of selenium, gold, silver, or heparin.

22. The device of claim 20, wherein the anti-fouling material includes polyisobutylene-co-polyurethane or polystyrene-co-isobutylene-co-styrene.

23. The device of claim 20, wherein the anti-fouling material is etched or textured to resist attachment by microorganisms or mucosubstances.
